(19) United States
(12) Patent Application Publication
(10) Pub. No.: US 2019/0206536 A1
(43) Pub. Date: Jul. 4, 2019

(54) SYSTEM AND METHOD FOR PRESCRIPTION MONITORING AND DRUG DISPENSATION UTILIZING A DISTRIBUTED LEDGER

(71) Applicant: Brian Hausman, Houston, TX (US)

(72) Inventor: Brian Hausman, Houston, TX (US)

(21) Appl. No.: 15/859,733

(22) Filed: Jan. 1, 2018

(51) Int. Cl. G16H 20/10; G16H 10/60; G16H 80/00; H04L 9/32

(52) U.S. Cl. CPC G16H 20/10; G16H 10/60; G06Q 50/26; H04L 9/3226; G16H 80/00

(57) ABSTRACT

The present disclosure relates to implementing an electronic prescription and drug system in a decentralized computing network that employs a distributed ledger. A method for prescribing and dispensing drugs may comprise of examining a patient; checking an electronic prescription and drug system, wherein the electronic prescription and drug system comprises a plurality of nodes, wherein the plurality of nodes comprise a processor, a memory unit, and a bus, wherein the plurality of nodes are connected to each other over a communication network, wherein each of the plurality of nodes has access to a copy of a distributed ledger, wherein the plurality of nodes verify and record transactions occurring within the distributed ledger; administering a prescription; dispensing a drug; and broadcasting transactions to the distributed ledger.

### BACKGROUND OF THE INVENTION

Medical prescriptions, referred to herein as “prescriptions,” may be instructions given to a patient concerning the patient's plan of care. A prescription may be administered by a physician or other health care provider. Typically, the prescription is the written authorization for a patient to purchase a prescription drug from a pharmacist. Recently, the United States has implemented prescription monitoring programs (PMPs) and prescription drug monitoring programs (PDMPs). Both of these programs are state-run which collect and distribute data about the prescription and dispensation of federally controlled substances and, as the individual states deem appropriate, other potentially addictive or abusable prescription drugs. Misuse or abuse of prescription drugs may lead to addiction, and in some cases death, of a patient.

These PMPs and PDMPs may use electronic databases to track and store the dispensation and fulfillment of prescriptions. They may collect, monitor, and analyze electronically transmitted prescribing and dispensing data submitted by pharmacies or health care providers. The data may be used to support states' efforts in education, research, enforcement, and abuse prevention. However, there are drawbacks to these programs. Most programs are state-run. Therefore, each state's program(s) may be different from others, Each state may have different requirements for the programs. For example, some states may require both the health care provider writing the prescription and the pharmacist fulfilling it, only the health care provider, only the pharmacist, or neither to enroll in the program. The frequency of data collection may be different per program. Some states may require data to be collected daily, weekly, or monthly.

Health insurance plans and malpractice insurance plans may also have a stake in monitoring prescription writing and prescription drug dispensation. Health insurance plans may pay a portion, if not all, of the cost of certain medical expenses, including prescription drugs. Misuse or abuse of prescription drugs may increase the cost to health insurance plans as individual patients may use their health insurance plans to pay for the prescription drugs. There may be mistakes in the initial writing of a prescription, the interpretation of the prescription by a pharmacist, or in entering in the detailed information in databases. Health care providers and institutions may be held liable for mistakes.

Doctor shopping may be inhibited through a closer inspection of the process of acquiring prescription drugs. Often, a patient may visit multiple health care providers in order to collect multiple prescriptions for certain drugs. This may occur if the patient is an addict or is trying to sell the drugs for a profit. Doctor shopping is illegal, and law enforcement may become involved. Law enforcement may not have access to hospital databases or a patient's medical records. Enforcing the appropriate punishment for doctor shopping may be difficult or time-consuming with current methods and systems in place.

Thus, there is a need for a system and method to monitor the supply of prescriptions and the dispensation of drugs.

### BRIEF DESCRIPTION OF THE DRAWINGS

For a detailed description of the preferred examples of the invention, reference will now be made to the accompanying drawings in which:

FIG. 1 illustrates an example of a decentralized computing network;

FIG. 2 illustrates an example of a blockchain; and

FIG. 3 illustrates an example of a flowchart utilizing an electronic prescription and drug system.

### DETAILED DESCRIPTION OF THE PREFERRED EXAMPLES

The present disclosure relates to implementing an electronic prescription and drug system in a decentralized computing network that employs a distributed ledger. More particularly, examples of a system and method may be disclosed for processing the initial supply of a prescription and the dispensation of a drug upon fulfillment of that prescription. The decentralized computing network may include a plurality of computing systems that act as nodes. Each node may access the distributed ledger. Without limitation, the distributed ledger may utilize blockchain technology and protocols, directed acyclic graph (DAG) technology and protocols, and/or combinations thereof.

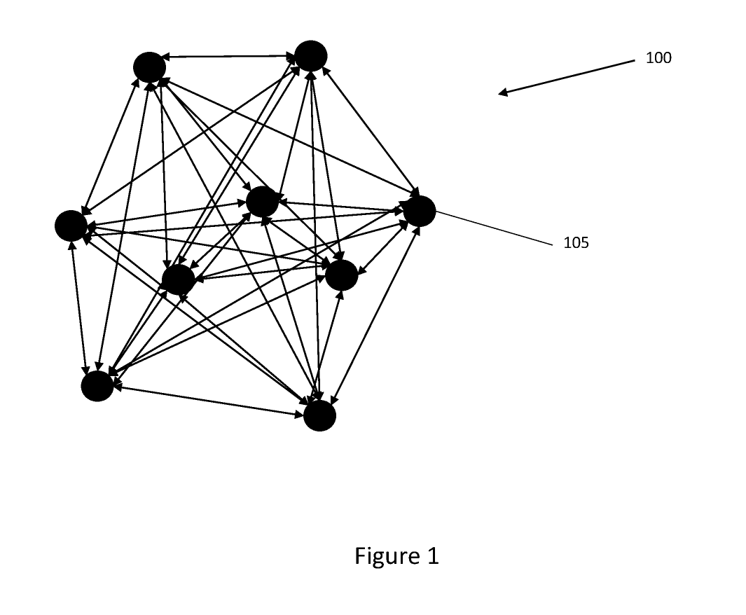

FIG. 1 illustrates an example of a decentralized computing network 100. Decentralized computing network 100 may include a plurality of nodes 105. Node 105 may be operated by an individual, company, and/or other entity. Each node 105 may include a processor, a memory unit, a power supply, a bus, and/or combinations thereof. The memory unit may be volatile and/or non-volatile. Further hardware and/or software may be used by each node 105. Additionally, any suitable input and output (I/O) devices may be implemented. Without limitation, node 105 may be a computer and/or laptop. Concerning the present disclosure, computer-readable storage mediums may be utilized.

Decentralized computing network 100 may connect the plurality of nodes 105 by any form or medium of digital data communication such as a communication network. Without limitation, a communication network may include a local area network (“LAN”), a metropolitan area network (“MAN”), a wide area network (“WAN”), peer-to-peer networks (structured, unstructured, and/or hybrid models), grid computing infrastructures, the Internet, and/or combinations thereof.

In examples, decentralized computing network 100 may utilize blockchain technology and protocols for the distributed ledger. However, not all distributed ledgers may necessarily employ blockchain technology to successfully provide secure and valid achievement of distributed consensus. Without limitation, a blockchain may be one type of data structure considered to be a distributed ledger. A blockchain may be a continuously growing list of records. In examples, the records may be represented as blocks. Each block may include transaction data, a hash pointer, a timestamp, and/or combinations thereof. These blocks may be linked and secured using cryptographic measures. Cryptographic measures may include any suitable mathematical algorithm. In examples, a hash function may be used as the cryptographic measure, wherein the hash function is a mathematical algorithm that takes a data input and generates a fixed output (e.g., a bit string with a fixed length). Hash functions may have pre-image resistance, wherein it may be infeasible to invert without using a brute-force method of trying to compare hashed values of random inputs. Hash functions may be collision resistant, wherein it may be infeasible for two given inputs to produce the same output. In examples, a hash function may be designed to be a one-way function.

The methodology behind blockchain may promote a decentralized network over a peer-to-peer network rather than a central computing system. In examples, the plurality of nodes 105 may own a full copy of the distributed ledger. When a transaction occurs in the distributed ledger, the plurality of nodes 105 may verify the status of their respective copy of the distributed ledger (i.e., the addition of a new block). A consensus among the plurality of nodes 105 may be required to verify the status of the distributed ledger. Any suitable protocol may be used to reach consensus. Without limitation, suitable protocols may be proof of work, proof of stake, proof of authority, and/or combinations thereof. In examples, this may occur automatically and/or continuously. Once consensus has been reached, the distributed ledger may be updated (i.e., the addition of a block). In examples, any suitable data mining technique may be used to verify and/or create the addition of a block in a blockchain. Nodes 105 may be incentivized to verify and/or create a new block in the blockchain with a reward.

In examples, digital signatures may be used in the blockchain. In examples, a public and private key may be created using an algorithm and may be related to each other. The public key may be distributed to the plurality of nodes 105. The private key may be kept by an individual node 105 to digitally sign any transaction occurring in the distributed ledger. The receiving party of a transaction that has been signed may verify the data within the transaction by using the public key. One of ordinary skill in the art would recognize that any known digital signature systems may be used without departing from the spirit and scope of the present invention.

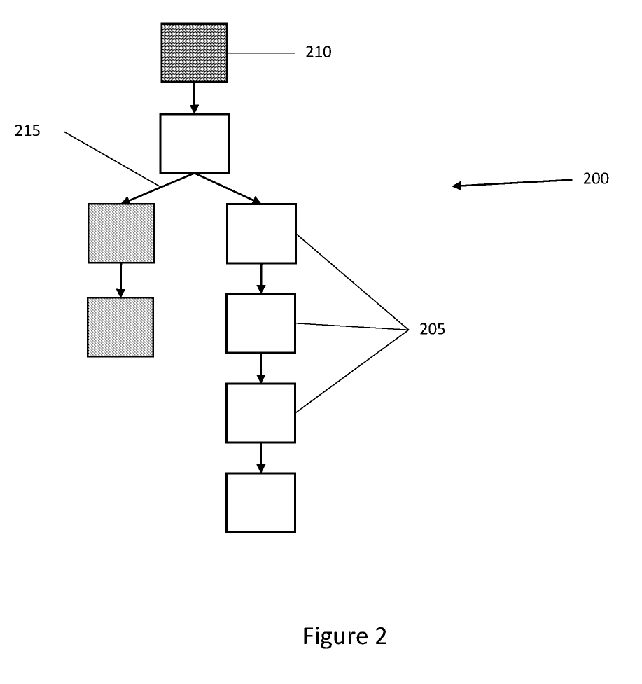

FIG. 2 illustrates an example of a blockchain 200. There may be a plurality of blocks 205 within blockchain 200. In examples, a first block 210 may represent the first data transactions within the distributed ledger. The first block 210 may include any suitable size of data. A hash function may be used to generate an output value (e.g., a “hash”) from the first data transactions. For each subsequent block 205 added to blockchain 200, the input to the hash function of the new block may include the previous block's hash and the data transactions represented by the new block. This may produce a system wherein the plurality of blocks 205 are linked, in sequential order, by the previous block's output value of the hash function. The linked blocks 205 may allow the plurality of nodes 105 (referring to FIG. 1) to follow blockchain 200 backwards, from progression, in order to observe and verify data transactions.

In examples, a fork 215 may be created within blockchain 200. This may be when blockchain 200 diverges into two potential paths of progression. Without limitation, fork 215 may be introduced when two blocks 205 are added that claim the hash of the previous block, when an invalid transaction occurs, and/or when new protocols are implemented. In the example wherein fork 215 is introduced due to two blocks being added, a portion of the plurality of nodes 105 (referring to FIG. 1) may allocate computational efforts in adding blocks onto one side of fork 215. The remaining portion of the plurality of nodes 105 may allocate computational efforts in adding blocks onto the other side of fork 215. One side of fork 215 will inevitably surpass the other in length. The plurality of nodes may come to an agreement that the longer side of fork 215 is the legitimate path of progression of blockchain 200. In examples, the path of progression on the other side of fork 215 may be deemed invalid, may be abandoned, and/or data transactions within the blocks may be lost. In examples with accordance to this disclosure, blockchain 200 may be able to accommodate forks.

In examples, the distributed ledger may be private, public, and/or combinations thereof. The electronic prescription and drug system may have access to information that a patient of a health care provider may not want publicly disclosed. There may be different types of nodes 105 (referring to FIG. 1) in the electronic prescription and drug system. There may be limited access within the distributed ledger based on the type of node 105 in operation. Without limitation, different types of nodes 105 that may interact within the electronic prescription and drug system may represent individual health care providers, hospitals, insurance companies, pharmacies, other companies, the general public, the government, and/or combinations thereof. In examples, the distributed ledger may be shared and/or compatible with distributed ledgers and/or databases belonging to other entities. This may allow cross-referencing between entities.

In examples, smart contracts may be used within the distributed ledger. Smart contracts may be computer protocols to execute the agreed upon terms of a contract. Smart contracts may be partially and/or fully self-executing. In examples, smart contracts may be written as code in blockchain 200. Transactions within a smart contract may be reflected in blockchain 200 as the plurality of nodes 105 (referring to FIG. 1) receive the data transactions, verify the information, and update their respective copies of the distributed ledger.

In alternative examples, there may be disadvantages to implementing blockchain technology and protocols for the electronic prescription and drug system. Transaction times and data mining fees may be variables in the overall usage of the electronic prescription and drug system. Directed acyclic graph (DAG) technology and protocols may be an alternative way to monitor the electronic prescription and drug system. The distributed ledger may implement DAG technology and protocols over decentralized computing network 100 (referring to FIG. 1).

DAGs may be finite directed graphs with no directed cycles. That is, they consist of finitely many vertices and edges, with each edge directed from one vertex to another, such that there is no way to start at any vertex and follow a consistently-directed sequence of edges that eventually loops back to the same vertex again. Equivalently, a DAG is a directed graph that has a topological ordering, a sequence of the vertices such that every edge is directed from earlier to later in the sequence. In examples, a transaction, data set, representation of information, and/or combinations thereof may represent a vertex within a DAG. A singular node 105 (referring to FIG. 1) may broadcast or upload this vertex to the DAG. Without limitation, an edge may form between the broadcasted or uploaded vertex and at least one previous vertex within the DAG. In examples, this edge may form by approving the validity of that previous vertex within the DAG. In examples, vertices may need to be connected to other vertices through edges. Otherwise, they may not be recorded within the distributed ledger. This may represent a form of proof-of-work for verifying vertices (i.e. transactions) within the distributed ledger. Approval may require computational time and effort and/or may be automatic. There may be vertices forming edges between any suitable number of previous vertices. In examples, DAG technology and protocols may provide a scalable distributed ledger based on the variable inputs.

Furthermore, the electronic prescription and drug system may implement a combination of blockchain and DAG technology and protocols.

In examples, a health care provider may operate as a node 105 (referring to FIG. 1) within the electronic prescription and drug system. Without limitation, a health care provider may be a physician, physician's assistant, nurse, psychiatrist, dentist, optometrist, or a veterinarian. Health care providers may work at hospitals, healthcare centers, academic institutions, research institutions, and/or in administration. In examples, the entire entity regulating the health care provider's place of work may operate as a node 105 (referring to FIG. 1) within the electronic prescription and drug system.

A patient may approach a health care provider requesting treatment. In examples, the health care provider may recommend a treatment plan involving prescription drugs. The health care provider may administer a prescription for the patient to be filled at a pharmacy. Typically, a pharmacist may be able to fill the prescription. The prescription may be hand-written on pre-printed forms. In examples, the pre-printed forms may have been assembled into pads. In other examples, the prescription may be administered orally between the health care provider and a pharmacist. In alternate examples, the prescription may be entered into an electronic medical record system and transmitted electronically to a pharmacy.

Once the administered prescription has been delivered to a suitable pharmacy, the pharmacist may fill the prescription and dispense the requisite drug(s). Without limitation, a suitable pharmacy may be any favorable establishment that prepares and dispenses drugs (i.e. favorable factors may include distance from a location, cost, time, etc.). The patient may pay for the drug and commence in the recommended treatment process.

The process described above may be prone to error and inefficiencies. Often, either the health care provider and/or the pharmacist may have to enter in the prescription information into a computer for online storage. This may lead to mistakes made in the dispensation and/or application of powerful drugs. Concerning the present disclosure, an electronic prescription and drug system may be implemented by health care providers, pharmacists, hospitals, pharmacies, insurance companies, law enforcement, and/or combinations thereof.

In examples, the process of administering a prescription, getting the prescription filled, and dispensing drugs may be entirely or partially done within the electronic prescription and drug system. Decentralized computing network 100 (referring to FIG. 1) may have access to a distributed ledger, wherein the distributed ledger may be capable of recording all data transactions uploaded to decentralized computing network 100 by the plurality of nodes 105 (referring to FIG. 1). Without limitation, data transactions may include any suitable prescription information, any suitable information pertaining to the action of administering a prescription, delivering the prescription to a pharmacy, any suitable pharmacy information, filling the prescription, dispensing the requisite drug for the prescription, the payment for the dispensation of the drug and/or combinations thereof.

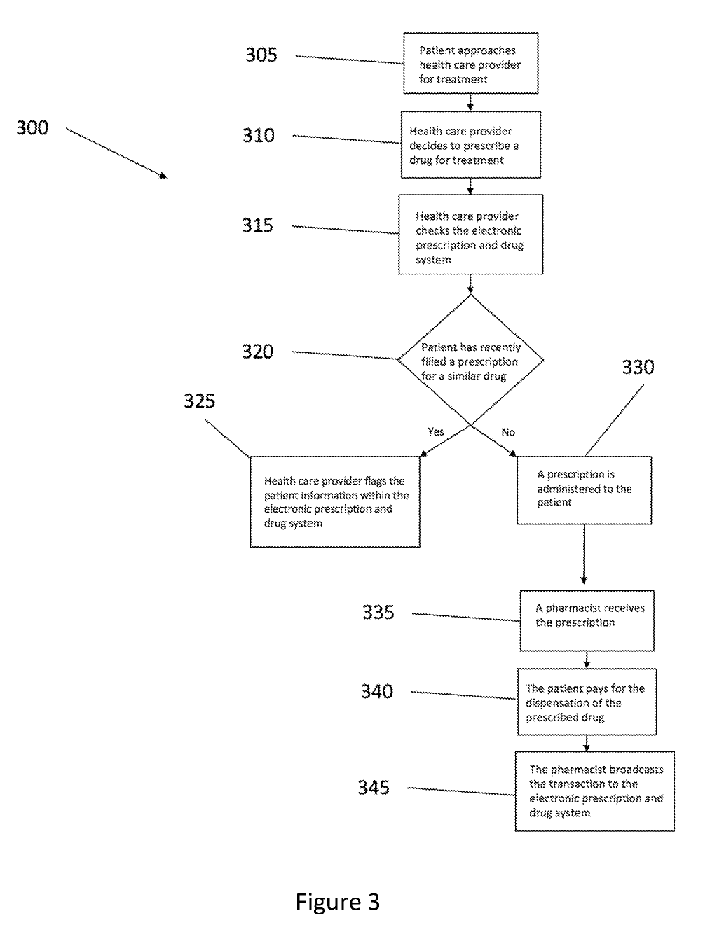

FIG. 3 illustrates an example of a flowchart 300 which may show the process of utilizing the electronic prescription and drug system. The first step for flowchart 300 may begin with step 305. Step 305 may include a patient approaching a health care provider for treatment. In examples, this may occur in a hospital and/or a doctor's office. The patient may be experiencing pain, discomfort, sickness, and/or combinations thereof. The health care provider may conduct any suitable tests to determine the cause of the patient's pain, discomfort, sickness, and/or combinations thereof and may relieve any symptoms.

In step 310, the health care provider may decide to prescribe a drug as a part of a treatment for the patient. This may occur after the health care provider has determined the cause of the patient's pain, discomfort, sickness, and/or combinations thereof In other examples, the health care provider may not have been able to determine the cause of the patient's pain, discomfort, sickness, and/or combinations thereof. More tests and/or examination procedures may be required prior to coming to the conclusion of a treatment.

The health care provider may check the electronic prescription and drug system in step 315. This may occur at any time prior to administering a prescription to the patient. Without limitation, this may occur when the patient initially schedules an appointment with the health care provider, when the patient arrives for the appointment, after tests and/or examination procedures have been conducted between the patient and the health care provider, and/or combinations thereof.

The health care provider may collect information before, during, and/or after the appointment to create and/or edit the patient's profile. The patient's profile may be recorded and stored by the health care provider and/or the health care provider's place of work. Without limitation, the patient's profile may include information such as current residence, contact information, background medical history, and/or combinations thereof. This information may have been uploaded or broadcasted to the decentralized computing network 100 (referring to FIG. 1). A plurality of nodes 105 (referring to FIG. 1) may have verified the uploaded or broadcasted patient's profile. Verification may have been done using a proof of work, proof of stake, proof of authority, and/or combinations thereof. The information within the patient's profile may be represented as a block. The block may be encrypted and added to the blockchain within the electronic prescription and drug system.

In examples, the electronic prescription and drug system may be distributed across decentralized computing network 100 (referring to FIG. 1). The health care provider may access the electronic prescription and drug system as a node 105 (referring to FIG. 1). The health care provider may be authorized to have full access within the electronic prescription and drug system. In alternate examples, the health care provider may have limited access. Without limitation, the limited access may include the health care provider only being able to view the information within the electronic prescription and drug system (i.e., the health care provider cannot broadcast or upload information to the system), broadcasting certain information, being able to view the health care provider's specific patients' information, flagging or categorizing a patient's information within the electronic prescription and drug system, and/or combinations thereof.

Step 320 may include a logical decision split within flowchart 300. Depending on the information pertaining to the question prompted in step 320, the health care provider may take different actions. The health care provider may determine whether or not the patient has recently filled a prescription for the same and/or similar drug that the health care provider has recommended as a treatment. There may be rules set by the health care provider's place of work specifying a required minimum time a patient has to wait before being prescribed the same and/or similar drug. There may be local, state, and or federal laws in place also specifying a required minimum time a patient has to wait before being prescribed the same and/or similar drug. The health care provider may be able to parse through the blockchain of the electronic prescription and drug system to search for the patient's profile. In examples, this may include de-encrypting a block, or blocks, within the blockchain to gain access to the patient's profile. The patient's profile may have to be extracted from a block. The patient's profile may contain information such as history of prescribed drugs, frequency of prescriptions, location of filled prescription and drug dispensation, and/or combinations thereof. Once the health care provider has access, the health care provider may make a decision based off of the patient's profile.

If there is information within the electronic prescription and drug system that suggests that the patient has recently filled a prescription for the same and/or similar drug, the health care provider may flag the patient's profile within the electronic prescription and drug system, as seen in step 325. The action of flagging the patient's profile may be seen as changing the blockchain within the electronic prescription and drug system. This may be broadcasted over the decentralized computing network 100 (referring to FIG. 1). The plurality of nodes 105 (referring to FIG. 1) may verify the patient's profile as “flagged” and update their respective copies of the distributed ledger. The health care provider may refuse to prescribe the initial prescription drug. The health care provider may refuse any additional service and/or treatment to the patient. In other examples, the health care provider may recommend prescribing a different drug.

In examples, law enforcement and/or the government may have access to the electronic prescription and drug system. They may have limited and/or full access within the electronic prescription and drug system (provided legal pre-requisites are met). Law enforcement and/or the government may operate as nodes 105 (referring to FIG. 1) within the decentralized computing network 100 (referring to FIG. 1). In examples, they may operate within the terms of a smart contract. Without limitation, the terms within the smart contract may allow the law enforcement and/or government nodes 105 to have access to the flagged patient profiles. They may conduct research and study the flagged patient profiles for potential persons whom have broken the law and/or are about to break the law.

If there is information within the electronic prescription and drug system that does not suggest that the patient has recently filled a prescription for the same and/or similar drug, the prescription may be administered to the patient, as seen in step 330. As previously discussed, there may be numerous ways for the health care provider to administer a prescription. Concerning the present disclosure, the health care provider may administer the prescription through the electronic prescription and drug system. In examples, the health care provider may input the prescription information into the patient's profile. The health care provider may broadcast or upload the patient's profile, with the new prescription information, to the decentralized computing network 100 (referring to FIG. 1). The plurality of nodes 105 (referring to FIG. 1) may verify the patient's profile containing the prescription information and update their respective copies of the distributed ledger.

In examples, the patient may choose, while at the appointment, which pharmacy to have the prescription filled. The health care provider may enter into a smart contract with that specific pharmacy. In alternate examples, the patient may not know which pharmacy to choose to have the prescription filled. The health care provider may enter into a more general smart contract requiring the other entity to be a pharmacy. This may provide the patient with the flexibility to go to any pharmacy to fulfill the prescription. The health care provider may broadcast or upload all suitable information regarding the prescription administered to the patient into the smart contract. The plurality of nodes 105 (referring to FIG. 1) may verify the creation of the smart contract and update their respective copies of the distributed ledger.

Step 335 may include a pharmacist receiving the prescription administered to the patient by the health care provider. There may be a variety of ways that the pharmacist may receive the prescription administered to the patient. In examples, the patient may enter into a pharmacy and give the pharmacist a written prescription. In other examples, the pharmacist may be informed, either by the patient or the health care provider, that the patient has a prescription administered through the electronic prescription and drug system. The pharmacist may access the electronic prescription and drug system as a node 105 (referring to FIG. 1). The pharmacist may be authorized to have full access within the electronic prescription and drug system. In alternate examples, the pharmacist may have limited access. Without limitation, the limited access may include the pharmacist only being able to view the information within the electronic prescription and drug system (i.e., the pharmacist cannot broadcast or upload information to the system), broadcasting certain information, and/or combinations thereof. In examples, the pharmacist may be able to parse through the blockchain of the electronic prescription and drug system to search for the patient's profile. In examples, this may include de-encrypting a block, or blocks, within the blockchain to gain access to the patient's profile. The pharmacist may be able to access the prescription if the prescription had been broadcasted with the patient's profile. In alternate examples, the pharmacy may be the designated party for a smart contract. The pharmacist employed by the pharmacy may represent the pharmacy and enter into the smart contract as the designated party. The pharmacist may receive the prescription information. There may be terms within the smart contract that the pharmacist may have to fulfill. In other examples, the pharmacy may not be the designated party for a smart contract, but the prescription information is within the smart contract. The smart contract may require that the missing party be a pharmacy in order to allow that party to enter into the contract. Once the pharmacist, acting on behalf of the pharmacy, enters into the smart contract, methods as previously described may allow the pharmacist to receive the prescription information.

Once the pharmacist has all the suitable information concerning the prescription, the pharmacist may proceed to fill the prescription. In examples, the patient may be alerted that the prescription has been filled. The patient may pay the pharmacist for the dispensation of the prescribed drug, as seen in step 340.

Step 345 may include the pharmacist broadcasting or uploading the payment transaction to the electronic prescription and drug system. The payment transaction information may be detailed or simple. In examples, the payment transaction information may be a confirmation whether or not the patient paid for the prescription drug. Alternatively, without limitation, the payment transaction information may include the monetary amount, the time and date of the transaction, method of payment, prescription drug information, and/or combinations thereof. The plurality of nodes 105 (referring to FIG. 1) may verify the payment transaction and update their respective copies of the distributed ledger.

In examples, the distributed ledger may be a single or multiple blockchains 200 (referring to FIG. 2). Alternatively, the distributed ledger may use variants of blockchain, including DAG technology and protocols, that may maintain the decentralized network and the cryptographic measures. The distributed ledger may be compatible with other entities' databases. In examples, a pharmacy may be able to use the distributed ledger with its own database (which may contain drug inventory and delivery schedules) when doing business with a patient. In examples, the distributed ledger may provide a reliable system that insurance companies may be able to reference in instances of malpractice. The distributed ledger may be able to monitor a health care provider's prescribing habits, which may include the frequency at which a specific drug is prescribed and/or the frequency at which a specific patient is administered a prescription.

Although the present invention and its advantages have been described in detail, it should be understood that various changes, substitutions and alterations may be made herein without departing from the spirit and scope of the invention as defined by the appended claims.

What is claimed is:

1. A method for prescribing and dispensing drugs, comprising:
checking an electronic prescription and drug system, wherein the electronic prescription and drug system comprises:
a plurality of nodes, wherein the plurality of nodes comprise a processor, a memory unit, and a bus, wherein the plurality of nodes are connected to each other over a communication network, wherein each of the plurality of nodes has access to a copy of a distributed ledger, wherein the plurality of nodes verify and record transactions occurring within the distributed ledger;
administering a prescription;
dispensing a drug; and
broadcasting transactions to the distributed ledger.

2. The method of claim 1, wherein the electronic prescription and drug system displays a patient's profile, wherein the patient's profile contains background medical history, current residence, and contact information.

3. The method of claim 2, further comprising:
examining a patient; and
a health care provider flagging the patient's profile.

4. The method of claim 2, wherein the administering a prescription comprises of broadcasting the prescription with the patient's profile over the communication network.

5. The method of claim 2, wherein the administering a prescription comprises of entering into a smart contract with a pharmacy, wherein the pharmacy receives the prescription by agreeing to the terms of the smart contract.

6. The method of claim 1, wherein the broadcasting transactions to the distributed ledger includes payment information and the prescribed drug information.

7. The method of claim 1, wherein the electronic prescription and drug system uses blockchain technology and protocols.

8. The method of claim 1, wherein the electronic prescription and drug system uses directed acyclic graph technology and protocols.

9. An electronic prescription and drug system, comprising:
a plurality of nodes, wherein the plurality of nodes comprise a processor, a memory unit, and a bus, wherein the plurality of nodes are connected to each other over a communication network, wherein each of the plurality of nodes has access to a copy of a distributed ledger, wherein the processor of each of the plurality of nodes is configured to:
verify and record transactions occurring within the distributed ledger, wherein the transactions are encrypted by a mathematical formula that produces a hash value, wherein each transaction is linked to another transaction by the hash value of the previous transaction, wherein a consensus must be reached to update the distributed ledger with the addition of a new transaction.

10. The electronic prescription and drug system of claim 9, wherein the plurality of nodes utilize blockchain technology and protocols.

11. The electronic prescription and drug system of claim 9, wherein the plurality of nodes utilize directed acyclic graph technology and protocols, wherein transactions are represented as vertices, wherein edges form by approving other previous transactions.

12. The electronic prescription and drug system of claim 9, wherein the plurality of nodes verify the transactions by a protocol employing proof-of-work, proof-of-stake, or proof-of-authority to reach consensus.

13. The electronic prescription and drug system of claim 9, wherein there is limited access based on the operating entity of each node.

14. The electronic prescription and drug system of claim 9, wherein the plurality of nodes use smart contracts for transactions, wherein the smart contracts are self-executing.

15. The electronic prescription and drug system of claim 9, wherein the mathematical formula is a hash function, wherein the hash function produces the hash value, wherein the hash value is a bit string with a fixed length.

16. The electronic prescription and drug system of claim 9, wherein a health care provider broadcasts a prescription over the communication network.

17. The electronic prescription and drug system of claim 9, wherein a pharmacist accesses the prescription over the communication network.

18. The electronic prescription and drug system of claim 9, wherein the pharmacist broadcasts the dispensation of a prescribed drug over the communication network.

19. A method for prescribing and dispensing drugs, comprising:
examining a patient;
checking an electronic prescription and drug system, wherein the electronic prescription and drug system uses blockchain technology and protocols, wherein the electronic prescription and drug system displays a patient's profile, wherein the patient's profile contains data, wherein the data is background medical history, current residence, contact information, or combinations hereof, wherein the electronic prescription and drug system comprises:
a plurality of nodes, wherein the plurality of nodes comprise a processor, a memory unit, and a bus, wherein the plurality of nodes are connected to each other over a communication network, wherein each of the plurality of nodes has access to a copy of a distributed ledger, wherein the plurality of nodes verify and record transactions occurring within the distributed ledger;
administering a prescription;
dispensing a drug; and
broadcasting transactions to the distributed ledger
